(19) United States
(12) Patent Application Publication
(10) Pub. No.: US 2021/0299381 A1
(43) Pub. Date: Sep. 30, 2021

(54) Surgical Device For Creating A Surgical Airway

(71) Applicant: Robert Snow, Clemmons,, NC (US)

(72) Inventor: Robert Snow, Clemmons,, NC (US)

(21) Appl. No.: 16/833,661

(22) Filed: Mar. 29, 2020

(51) Int. Cl. A61M 16/04

(52) U.S. Cl. CPC A61M 16/0472

(57) ABSTRACT

A surgical device used for creating a surgical airway into the patient's trachea is disclosed. The device could enable a person or medical professional to make an incision through the patient tissue for creating the surgical airway in the patient's trachea. The device comprises a handle and a bolster. The handle having a longitudinal cavity at a second end for securely receiving and holding a blade. The bolster is securely and slidably positioned into a recess at the second end of the handle using a pair of latches. The bolster is configured to freely slide in and out of the handle for concealing and exposing the blade, thereby enabling a person or medical professional to make an incision proximate the patient's trachea for creating the surgical airway and allowing to simply insert a conventional tracheal tube via the incision to facilitate oxygenation and ventilation during emergency medical conditions.

### CROSS REFERENCE TO RELATED APPLICATIONS

None.

### TECHNICAL FIELD

The present invention generally relates to the field of medical instruments such as surgical instruments and techniques for creating an opening in air passageways, and more particularly to a surgical device for making an incision through a patient tissue for creating a surgical airway in a patient's trachea, thereby simply inserting a tracheal tube via the incision through the tissue proximate the patient's trachea to facilitate oxygenation and ventilation during emergency medical conditions.

### BACKGROUND

In medical emergencies, there is an important need to access and control the patient's airway. A lack of oxygen to the patient, even for a brief period of time, may prove fatal. Patients who are inflicted with upper airway obstructions generally receive a surgical airway when non-invasive techniques fail to clear their upper airway. There are generally three common approaches for creating a surgical airway: needle tracheotomy, standard tracheotomy, and cricothyroidotomy (also known as cricothyrotomy).

Cricothyrotomy became established as the preferred method of surgical emergency management during the 1970s and early 1980s. Cricothyrotomy is a procedure of making a surgical incision using a scalpel blade through the skin proximate the patient's trachea and cricothyroid membrane for the placement of a breathing tube to establish an airway during certain life-threatening situations, such as airway obstruction by a foreign body, airway edema or other medical emergencies. It is easier to learn, quicker to perform and is overall a safer procedure for the emergency medical professional or practitioner who performs surgical airways very infrequently. Cricothyrotomy is performed by creating an opening in the cricothyroid membrane and inserting a tracheal tube.

The general procedure for surgical cricothyrotomy includes different steps. At one step, the medical professional could locate and identify the patient's cricothyroid membrane. At another step, a vertical or horizontal skin incision is made through the cricothyroid membrane using a scalpel blade. Further, at another step, the medical professional could enlarge the incision using a hemostat. At another step, the medical professional could stabilize the surgical opening using a tracheal hook and insert the tracheal tube to facilitate oxygenation and ventilation. This surgical procedure requires, at minimum, the following tools/instruments: scalpel blade for making the incision, hemostat to enlarge the incision, a tracheal hook to stabilize the surgical opening, and a tracheal tube to allow passage of air into the trachea.

However, the instruments/tools may be relatively large in size and are cumbersome to be easily carried by the medical professional and difficult to use during an emergency. The mechanics of handling the scalpel blade for making vertical or horizontal incisions, and then placing the tracheal hook in the right position for holding and stabilizing the trachea can be challenging during an emergency where there is little or no preparation time available. There exists a need to minimize the number of instruments/tools required for the medical professional and simplify the procedures involved, thus increasing the probability of saving the patient's life.

A device is described in U.S. Pat. No. 7,308,896 to Rafael Cruz, which discloses a device consists of a combination of a tracheal hook and a scalpel device used for creating an emergency surgical airway for a patient. The device includes a handle and a scalpel portion attached to one end of the handle which includes a blade for creating an incision. The tracheal hook includes at least one hook which is integrated to the handle or slidably attached to the handle.

Another invention is described in US20100089405 to Ross Johnson, et al., therein is a description of a method of creating an airway proximate to the patient's trachea using a surgical tool. The surgical tool includes a handle having a channel, a tracheal hook connected to the handle, and a scalpel positioned in the channel of the handle. The tracheal hook and the scalpel are removably attached to the handle or integrated to the handle. The surgical tool further includes a cap used for protecting the scalpel.

Another patent, U.S. Pat. No. 8,356,598, to Royce Rumsey, describes an apparatus and method used in tracheostomy and cricothyroidotomy procedures. The apparatus for providing a passage into the body includes a first slider unit and a second slider unit, which are operably connected to a slider track for the retractable deployment of a scalpel and a tissue spreader attached to the body. Further, the auto-retraction of the scalpel is achieved by the movement of the second slider unit.

Though the above-mentioned inventions and patents disclose surgical devices with a combination of a blade and hooks for creating the surgical airway, none of the them provide the features and related advantages of the presently disclosed subject matter. Notwithstanding the advancements made in the field of medical instruments, there is a need to provide a surgical device with optimal design features for creating an airway under any emergency medical circumstances that significantly improves the effectiveness of simply performing the medical procedures.

### SUMMARY

This summary is provided to introduce in simplified form concepts that are further described in the following detailed descriptions. This summary is not intended to identify key features or essential features of the claimed subject matter, nor is it to be construed as limiting the scope of the claimed subject matter.

The present invention relates to a surgical device for making an incision through a patient tissue for creating a surgical airway in a patient's trachea, thereby simply inserting a tracheal tube via the incision through the tissue proximate the patient's trachea to facilitate oxygenation and ventilation during medical emergency conditions.

In an embodiment, the device is configured to enable a person or medical professional to make an incision through a patient's tissue/skin proximate the patient's trachea, thereby simply inserting a tracheal tube via the incision through the tissue proximate the patient's trachea to facilitate oxygenation and ventilation during emergency medical conditions. In one embodiment, the device comprises a handle and a bolster. In one embodiment, the handle includes a first end and a second end. In one embodiment, the handle further comprises a longitudinal cavity for securely receiving and holding a blade. In one embodiment, the blade could be, but not limited to, a scalpel. In one embodiment, the blade is securely positioned within the longitudinal cavity of the handle. In one embodiment, the bolster is securely and slidably positioned into a recess or an opening of the handle at the second end.

In one embodiment, the bolster is configured to freely slide in and out of the handle for concealing and exposing the blade, thereby enabling a person or medical professional to make an incision proximate the patient's trachea for creating the surgical airway and allowing to simply insert a tracheal tube via the incision to facilitate oxygenation and ventilation during emergency medical conditions. The bolster could freely slide into the handle when the device is pushing against the patient's tissue, thereby exposing the blade forwardly of the handle to make the incision proximate the patient's trachea.

In one embodiment, the bolster further comprises one or more hooks. In one embodiment, the hooks are molded at, but not limited to, a top portion of the bolster. The hooks are configured to enable the person or medical professional to hold the patient's tissue for opening the incision proximate the patient's trachea by holding the handle in traction perpendicular to the patient's skin. In one embodiment, the hooks could be, but not limited to, distally fixed angle hooks. In one embodiment, the hooks could be, but not limited to, wedge-shaped protrusions. The hooks could be made from a flexible material, but not limited to, a plastic polymer or other suitable and a durable medical grade material typically used for tracheal hooks. In one embodiment, the device is made of a flexible material including, but not limited to, plastic. In other embodiment, the hooks could be aligned and shaped as a slightly protruding wedge, enabling the tissue to slide onto the crest of the wedge upon tissue incision.

In one embodiment, the bolster is slidably positioned within the recess at the second end of the handle using a pair of latches. In one embodiment, the bolster slidably moves into and out of the handle to expose and conceal the blade. The bolster retracts into the handle and exposes the blade to enable the person or medical professional for making an incision through the tissue proximate the patient's trachea to provide a surgical airway. The bolster could slide out of the handle for concealing the blade to prevent inadvertent damages while inserting a tracheal tube via the incision to facilitate oxygenation and ventilation during emergency medical conditions.

In one embodiment, the bolster further comprises a pair of latches. The bolster could be slidably positioned into the recess at the second end of the handle and secured by engaging the pair of latches to mating slots on both sides of the handle. In one embodiment, the bolster includes a shape, but not limited to, a tapered shape or a flared shape. In one embodiment, the bolster is made of, but not limited to, plastic.

In one embodiment, the handle further comprises mating slots on sidewalls for slidably receiving the pair of latches of the bolster. The pair of latches could prevent the bolster from falling out of the handle. In one embodiment, the handle could be, but not limited to, a solid or injection molded handle formed from any one of material including, but not limited to, plastic or other suitable medical grade materials. In one embodiment, the handle includes a shape, but not limited to, a tapered shape or a flared shape and/or any other suitable shapes.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 is a top view of a surgical device according to one embodiment of the present invention.

FIG. 2 is a side view of the surgical device according to one embodiment of the present invention.

FIG. 3 is a perspective view of a bolster retracted into the handle to expose a blade according to one embodiment of the present invention.

FIG. 4 is a cutaway sectional view of the handle of the surgical device according to one embodiment of the present invention.

FIG. 5 is a perspective view of the handle of the surgical device according to one embodiment of the present invention.

FIG. 6 is a cutaway sectional view of the bolster with the blade in a retracted position according to one embodiment of the present invention.

FIG. 7 is a cutaway sectional view of the bolster with the blade in an extended position according to one embodiment of the present invention.

FIG. 8 is a top perspective view of the bolster of the surgical device according to one embodiment of the present invention.

FIG. 9 is a front view of the bolster of the surgical device according to one embodiment of the present invention.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although the term “step” may be expressly used or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

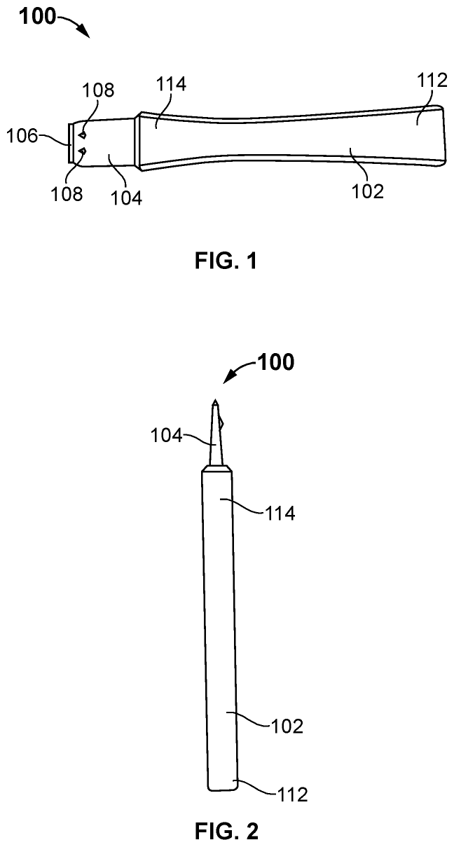

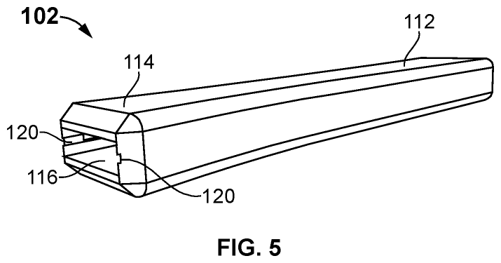

FIGS. 1 and 2 show a surgical device 100 used for providing a surgical airway in a patient's trachea according to one embodiment of the present invention. In an embodiment, the device 100 is configured to enable a person or medical professional to make an incision through a patient's tissue/skin proximate the patient's trachea, thereby simply inserting a tracheal tube via the incision through the tissue proximate the patient's trachea to facilitate oxygenation and ventilation during emergency medical conditions. In one embodiment, the device 100 comprises a handle 102 and a bolster 104. In one embodiment, the handle 102 includes a first end 112 and a second end 114. In one embodiment, the handle 102 further comprises a longitudinal cavity 118 (shown in FIG. 4) for securely receiving and holding a blade 106. In one embodiment, the blade 106 could be, but not limited to, a scalpel. In one embodiment, the bolster 104 is securely and slidably positioned into a recess or an opening 116 (shown in FIG. 5) of the handle 102 at the second end 114.

In one embodiment, the bolster 104 is configured to freely slide in and out of the handle 102 for concealing and exposing the blade 106, thereby enabling a person or medical professional to make an incision proximate the patient's trachea for creating the surgical airway and allowing to simply insert a tracheal tube via the incision to facilitate oxygenation and ventilation during emergency medical conditions. The bolster 104 could freely slide into the handle 102 when the device 100 is pushing against the patient's tissue, thereby exposing the blade 106 forwardly of the handle 102 to make the incision proximate the patient's trachea. In one embodiment, the bolster 104 further comprises one or more hooks 108. The hooks 108 could hold the patient's tissue for opening the incision proximate the patient's trachea for inserting the tracheal tube. In one embodiment, the device 100 is made of a flexible material includes, but not limited to, plastic.

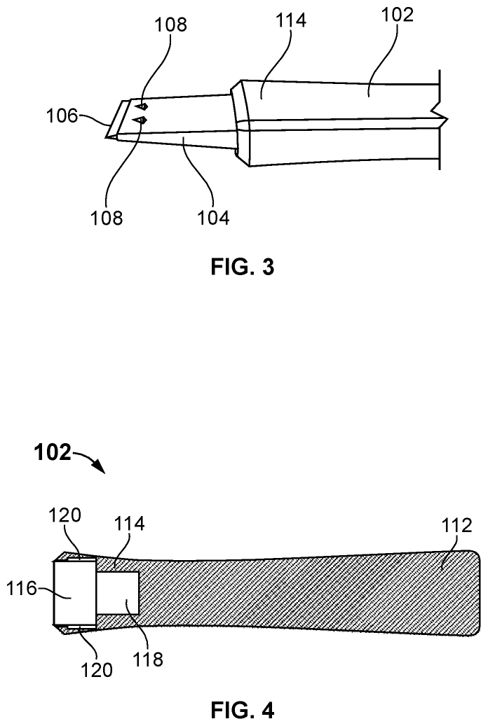

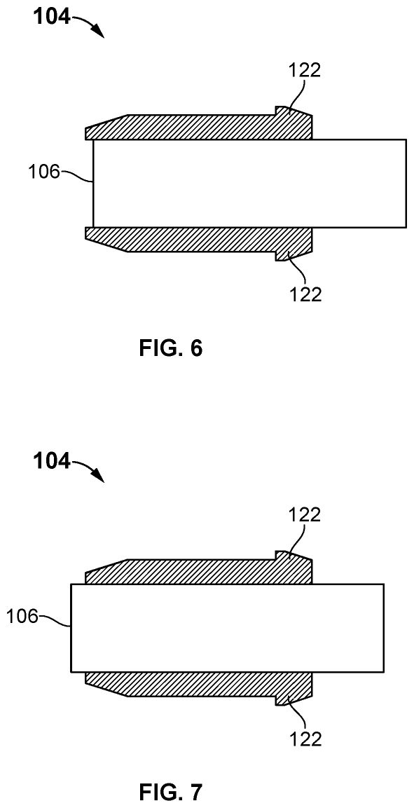

Referring to FIG. 3, the bolster 104 is retracted into the handle 102 to expose the blade 106 is disclosed. In one embodiment, the bolster 104 is slidably positioned within the recess 116 at the second end 114 of the handle 102 using a pair of latches 122 (shown in FIG. 6). In one embodiment, the hooks 108 are molded at, but not limited to, a top portion of the bolster 104. The hooks 108 are configured to hold the patient's tissue for stabilizing the surgical opening proximate the patient's trachea by holding the handle 102 in traction perpendicularly to the skin. In one embodiment the hooks 108 could be, but not limited to, distally fixed-angle hooks. In addition, the blade 106 could be made from, but not limited to, stainless steel or other suitable medical-grade materials typically used for surgical scalpel blades. The hooks 108 could be made from a flexible material, but not limited to, plastic or other suitable and a durable medical grade material typically used for tracheal hooks.

Referring to FIGS. 4 and 5, the handle 102 of the device 100 according to one embodiment of the present invention is disclosed. In one embodiment, the handle 102 having a passageway open/longitudinal cavity 118 at the second end 114 and substantially closed at the first end 112. The handle 102 could receive and secure the blade 106 (shown in FIG. 1) within the longitudinal cavity 118. In one embodiment, the handle 102 further comprises a recess 116 for slidably receiving the bolster 104. In one embodiment, the bolster 104 slidably moves into and out of the handle 102 to expose and conceal the blade 106. The bolster 104 retracts into the handle 102 and exposes the blade 106 to enable the person or medical professional for making incision through the tissue proximate the patient's trachea to provide a surgical airway. The bolster 104 could slide out of the handle 102 for concealing the blade 106 to prevent inadvertent damages while inserting a tracheal tube via the incision to facilitate oxygenation and ventilation during emergency medical conditions.

In one embodiment, the handle 102 further comprises mating slots 120 on sidewalls for slidably receiving the pair of latches 122 (shown in FIG. 6) of the bolster 104. The pair of latches 122 could prevent the bolster 104 from falling out of the handle 102 and prevent malalignment of the bolster 104 during usage of the device 100. In one embodiment, the handle 102 could be, but not limited to, a solid or injection molded handle formed from any one of material including, but not limited to, plastic or other suitable medical grade materials. In one embodiment, the handle 102 includes a shape, but not limited to, a tapered shape or a flared shape and/or any other suitable shapes. In some embodiments, the handle 102 could have textured patterns or grooves or ridges to facilitate the user to easily grip and handle the device 100.

FIG. 6 shows the blade 106 in a retracted position in one embodiment of the present invention, In one embodiment, the bolster 104 is configured to slide out of the handle 102 (shown in FIG. 1) for concealing the blade 106. In one embodiment, the bolster 104 further comprises a pair of latches 122. The bolster 104 could be slidably positioned into the recess 116 (shown in FIG. 5) at the second end 114 (shown in FIG. 5) of the handle 102 and secured by engaging the pair of latches 122 to mating slots 120 (shown in FIG. 5) on both sides of the handle 102. In one embodiment, the bolster 104 includes a shape, but not limited to, a tapered shape or a flared shape. In one embodiment, the bolster 104 is made of, but not limited to, plastic. FIG. 7 shows the blade 106 in an extended position in one embodiment of the present invention. In one embodiment, the bolster 104 is configured to slide into the recess 116 of the handle 102 for exposing the blade 106 when the device 100 is used to make an incision through the patient's tissue proximate the trachea.

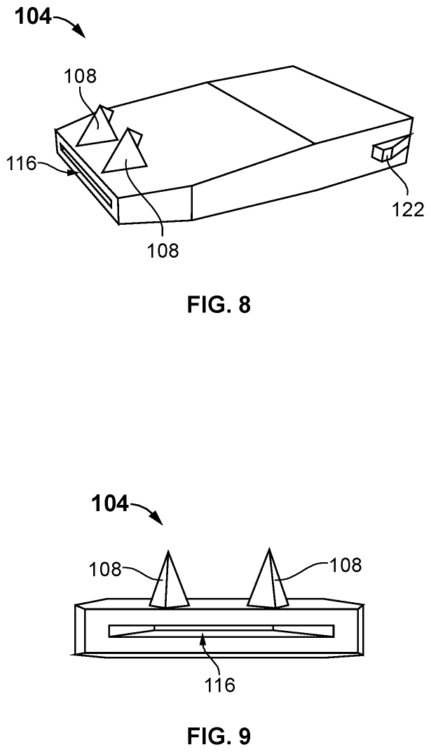

Referring to FIG. 8, the bolster 104 of the device 100 is disclosed in one embodiment of the present invention. In one embodiment, the bolster 104 further includes a tapered shape at one end and a pair of latches 122 on both sides at another end. In one embodiment, the pair of latches 122 engages to the mating slots 120 on both sides of the handle 102 to securely lock the bolster 104 to the handle 102. In one embodiment, the bolster 104 is made of a flexible material includes, but not limited to, plastic.

FIG. 9 shows the pair of hooks 108 on a top portion of the bolster 104 in one embodiment of the present invention. In one embodiment, the hooks 108 are configured to enable medical professional to hold the tissue and skin proximate the patient's trachea for opening the incision, thereby safely and simply inserting a tracheal tube via the incision to facilitate oxygenation and ventilation. The tracheal tube could aid the patient in breathing during medical emergencies. The device 100 is simple in design, easy to use and operate by a person or medical professional for making an incision through the patient's tissue proximate the trachea. The device 100 could allow the person or medical professional to insert any conventional tracheal tube through the incision proximate the patient's trachea.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

The invention claimed is:

1. A surgical device for creating a surgical airway into a patient's trachea, comprising:
a handle having a first end and a second end, wherein the second end comprises a longitudinal cavity for securely receiving and holding a blade;
a bolster securely and slidably positioned into a recess at the second end of the handle using a pair of latches, wherein the bolster is configured to freely slide in and out of the handle for concealing and exposing the blade, thereby enabling a person to make an incision into the patient's trachea for creating the surgical airway and allowing to simply insert a tracheal tube via the incision to facilitate oxygenation and ventilation during emergency medical conditions.

2. The surgical device of claim 1, wherein the bolster is configured to freely slide into the handle when the surgical device is pushing against a patient's tissue, thereby exposing the blade forwardly of the handle to make the incision proximate the patient's trachea for creating the surgical airway.

3. The surgical device of claim 1, wherein the bolster comprises one or more hooks.

4. The surgical device of claim 2, wherein the hook or hooks are molded at a top portion of the bolster, wherein the hooks are configured to hold the patient's tissue for opening the incision proximate the patient's trachea.

5. The surgical device of claim 2, wherein the hook or hooks are distally fixed-angle hooks.

6. The surgical device of claim 1, wherein the handle further comprises mating slots on both sides at the second end for securely and slidably receiving the pair of latches of the bolster.

7. The surgical device of claim 1, wherein the handle is made of polymer material.

8. The surgical device of claim 1, wherein the handle has a shape includes a tapered shape or a flared shape.

9. The surgical device of claim 1, wherein the pair of latches are configured to enable the bolster to slide in and out off the handle via the recess and mating slots at the second end and prevent the bolster from falling off the handle.

10. The surgical device of claim 1, wherein the bolster is made of a polymer material.

11. A surgical device for creating a surgical airway proximate a patient's trachea, comprising:
a handle having a first end and a second end, wherein the second end comprises a longitudinal cavity for securely receiving and holding a blade;
a bolster securely and slidably positioned into a recess at the second end of the handle using a pair of latches, wherein the bolster is configured to freely slide into the handle when the surgical device is pushing against a patient's tissue, thereby exposing the blade forwardly of the handle to make an incision proximate the patient's trachea for creating the surgical airway and allowing a person to simply insert a tracheal tube via the incision to facilitate oxygenation and ventilation during emergency medical conditions.

12. The surgical device of claim 11, wherein the bolster is further configured to conceal the blade by sliding out off the handle when the surgical device is used to hold the patient's tissue for opening the incision using a pair of hooks.

13. The surgical device of claim 12, wherein the pair of hooks are molded at a top portion of the bolster, wherein the pair of hooks are configured to hold the patient's tissue for opening the incision proximate the patient's trachea.

14. The surgical device of claim 12, wherein the pair of hooks are distally fixed-angle hooks.

15. The surgical device of claim 11, wherein the handle further comprises mating slots on both sides at the second end for slidably receiving the pair of latches of the bolster.

16. The surgical device of claim 11, wherein the handle is made of a polymer material.

17. The surgical device of claim 11, wherein the handle has a shape includes a tapered shape or a flared shape.

18. The surgical device of claim 11, wherein the bolster is made of a polymer material.

19. The surgical device of claim 11, wherein the pair of latches are configured to enable the bolster to slide in and out off the handle via the recess and prevent the bolster from falling off the handle.
